(19) United States
(12) Patent Application Publication
(10) Pub. No.: US 2021/0394209 A1
(43) Pub. Date: Dec. 23, 2021

(54) DRY FOGGING SYSTEM

(71) Applicant: Cory Brown, Farmington, UT (US)

(72) Inventor: Cory Brown, Farmington, UT (US)

(21) Appl. No.: 17/348,543

(22) Filed: Jun. 15, 2021

Related U.S. Application Data
(60) Provisional application No. 63/040,229, filed on Jun. 17, 2020.

(51) Int. Cl. B05B 7/24

(52) U.S. Cl. CPC B05B 7/241; A61L 2/22; B05B 7/2424; B05B 7/2421

(57) ABSTRACT

A dry fogging system includes a control device including a control device inlet configured to couple to a gas source, a first control device outlet, and a second control device outlet configured to couple to a first tank. The dry fogging system further includes a first spray device including a first spray device inlet configured to couple to the first tank, a second spray device inlet configured to couple to the first control device outlet, and a spray device outlet configured to provide dry fogging.

### RELATED APPLICATION

This application claims benefit of U.S. Provisional Patent Application 63/040,229, filed Jun. 17, 2020, the entire content of which is incorporated by reference herein.

### TECHNICAL FIELD

Embodiments of the present disclosure relate to a fogging system, and in particular to a dry fogging system.

### BACKGROUND

Some microorganisms and agents cause infectious diseases. Disinfection and sterilization of areas is used reduce or eliminate microorganisms and agents to provide a healthy environment.

### BRIEF DESCRIPTION OF THE DRAWINGS

The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 illustrates a system, according to certain embodiments.

FIG. 2 illustrates a control device of a system, according to certain embodiments.

FIG. 3 illustrates a compressor interface of a system, according to certain embodiments.

FIG. 4A illustrates a front perspective view of tanks of a system, according to certain embodiments.

FIG. 4B illustrates a top perspective view of a tank of a system, according to certain embodiments.

FIG. 4C illustrates a front cross-sectional view of a tank of a system, according to certain embodiments.

FIGS. 5A-B illustrate spray devices of a system, according to certain embodiments.

FIG. 6 illustrates a support structure of a system, according to certain embodiments.

FIG. 7 illustrates a flow diagram of a method of using a dry fogging system, according to certain embodiments.

### DETAILED DESCRIPTION OF EMBODIMENTS

Embodiments described herein are related to a dry fogging system.

Microorganisms (e.g., microscopic organisms) live in almost every habitat around the world. Pathogens (e.g., infectious agent, something that causes a disease) include infectious microorganisms and agents, such as virus (e.g., non-enveloped virus, enveloped virus), bacterium, protozoan, prion, viroid, and fungus. For example, some pathogenic bacteria cause diseases such as plague, tuberculosis, and anthrax. In another example, some protozoan parasites cause diseases such as malaria, sleeping sickness, dysentery, and toxoplasmosis. In another example, some fungi cause diseases such as ring worm, candidiasis, or histoplasmosis. Some pathogenic viruses cause influenza virus (e.g., the flu), yellow fever, severe acute respiratory syndrome coronavirus 2 (SARS-CoV-2) (e.g., coronavirus disease 2019 (COVID-19), novel coronavirus), and the like.

Some pathogens are spread via small droplets produced by coughing, sneezing, and talking. The droplets travel through the air and fall onto surfaces. Some pathogens are spread by an object (e.g., hand) contacting a surface. People can become infected by coming into contact with the droplets in the air or by touching a contaminated surface and then touching their face (e.g., eyes, nose, and/or mouth). In some instances, pathogens may be spread by an infected person before and while showing symptoms.

Some pathogens (e.g., the influenza virus) spread around the world in periodical outbreaks, resulting in millions of cases of severe illness and hundreds of thousands of deaths. Some pathogens have vaccines or specific antiviral treatments, while others do not. Pandemics (e.g., COVID-19) are a spread of a pathogen causing a disease across a large region, affecting a substantial number of people within a short period of time.

Conventionally, at least portion of the surfaces in an area are manually cleaned periodically (e.g., via vacuuming floors, dusting furniture, and/or sweeping floors). Conventionally, some surfaces are seldom or never cleaned. Manually cleaning uses much time, energy, and products (e.g., many disinfecting wipes). This causes different types of manual cleaning to only be performed periodically (e.g., nightly, weekly, monthly), if performed at all. Conventionally, manual cleaning of some surfaces a person using the space potentially touched is performed after the person using the space starts to have symptoms (e.g., desk, phone, keyboard, etc. are manually disinfected via disinfecting wipes). Conventional periodic manual cleaning does not remove all pathogens since an infected person can spread pathogens prior to showing symptoms, manually cleaning may not remove pathogens from all surfaces, and it may be unknown if an infected person used the area.

Some conventional systems include a cleaning system mounted to a vehicle (e.g., mounted to a truck or trailer) that includes one or more fixed reservoirs of cleaning and/or rinsing agent. A user pulls long hoses (e.g., chemical hose and air hose) from the vehicle-mounted cleaning system into a room in a building. Conventionally, the hoses are attached (e.g., taped together), are similar (e.g., same color, similar diameter), and are easily confused. The user returns to the vehicle to turn on the cleaning system, returns to the room in the building to provide a pressurized cleaning agent into the room, and continues travelling back and forth between the room and the vehicle to make adjustments (e.g., adjust pressure, etc.). To add more cleaning or rinsing agent to any of the reservoirs, the pressure is removed from the cleaning system (e.g., compressors are stopped), new cleaning or rinsing agent is added to the reservoirs, and the pressure is reapplied (e.g., compressors are started again). To change the cleaning agent being provided to the room, a rinsing agent is applied to the hoses (e.g., by stopping the compressors, connecting the hoses to a reservoir including the rinsing agent, and applying pressure by starting the compressors) to rinse the previous cleaning agent out of the long hoses. The vehicle-mounted cleaning system takes a lot of time and energy to clean a room by travelling back and forth between the room and the vehicle to startup the cleaning system and to adjust the cleaning system. The vehicle-mounted cleaning system wastes product to rinse out the long hoses. The vehicle-mounted cleaning system uses an increased amount of pressure and energy (e.g., higher capacity compressors) to pressurize the long hoses (e.g., air, cleaning agent, and/or rinsing agent in the long hoses). The vehicle-mounted cleaning system has more maintenance and replacement of parts due to multiple long hoses being pulled to different rooms in a building and due to the higher pressure. Turning off the compressors and restarting the compressors to add more cleaning agent and to change cleaning agent takes an increased amount of time and energy and decreases the life of the compressors. Going back and forth between the room and the vehicle to make adjustments to the cleaning system (e.g., change pressure, etc.) prevents rapid, precise control of the cleaning system.

Disadvantages of conventional manual cleaning and conventional vehicle-mounted systems cause increase of time, energy, and products used. This causes less surfaces to be cleaned (e.g., conventional manual cleaning is unable to clean all surfaces, conventional vehicle-mounted cleaning system is unable to clean all of the rooms) and allows the spread of pathogens. This is especially dangerous during periodical outbreaks (e.g., influenza virus) and pandemics (e.g., COVID-19).

The devices, systems, and methods disclosed herein provide a dry fogging system. The dry fogging system includes a control device, one or more tanks, and a spray device. The control device includes a first regulator, a first gauge coupled to the first regulator, a second regulator, and a second gauge coupled to the second regulator. The first regulator has a first inlet to receive pressurized gas from one or more compressors, a first outlet to provide first regulated pressurized gas to the second regulator, and a second outlet to provide second regulated pressurized gas to the spray device. In some embodiments, the first regulated pressurized gas and the second regulated pressurized gas are at approximately the same pressure that is lower than the pressure of the pressurized gas received by the first regulator. The second regulator has an inlet to receive the first regulated pressurized gas from the first regulator and has an outlet to provide third regulated pressurized gas to the tank. The third regulated pressurized gas provided to the tank is at a lower pressure than the second regulated pressurized gas provide to the spray device.

The tank includes walls that enclose an inner volume. The tank includes a lid configured to interface with an opening formed by the walls to provide a sealed environment in the inner volume of the tank. The tank is to receive liquid into the inner volume via the opening and then the opening is to be sealed by the lid. The tank includes an inlet (e.g., inlet post) disposed on a wall of the tank (e.g., upper wall) and a liquid outlet (e.g., liquid outlet post) disposed on a wall of the tank (e.g., upper wall). The inlet of the tank is to receive the third regulated pressurized gas from the outlet of the second regulator via a hose. The tank includes a gas outlet (e.g., gas outlet post). In some embodiments, the gas outlet is disposed on the lid. In some embodiments, the gas outlet is disposed on a wall of the tank (e.g., upper wall). The liquid outlet is coupled to a tube that is routed from the liquid outlet disposed on an upper wall of the tank to a location proximate the lower wall of the tank (e.g., the inlet and the gas outlet do not have a tube routed to a location in the tank).

The spray device includes a first inlet coupled to the second outlet of the first regulator via a hose to receive the second regulated pressurized gas from the control device. The spray device includes a second inlet coupled to the liquid outlet of the tank via a hose to receive pressurized liquid from the tank or the gas outlet of the tank via a hose to receive pressurized gas from the tank. The spray device includes an outlet (e.g., spray nozzle, atomizing head, etc.) to provide pressurized liquid (e.g., atomized liquid).

The control device, tank, and spray device are configured to be carried (e.g., via a support structure, via a carrier, via a backpack, etc.) to an area to be dry fogged which provides for shorter piping between the control device, tank, and spray device. The dry fogging system provides mobile sterilization through fog (e.g., pressurized liquid, atomized liquid). Different chemical agents (e.g., chemicals, chemical products, cleaning product, etc.) are more effective (e.g., for given purposes) when applied at particular levels of moisture (e.g., specific particle sizes). The fogging output of the dry fogging system of the present disclosure can be quickly changed between drier and wetter fog by controlling the amounts (e.g., pressures) of liquid and gas to the spray device.

In some embodiments, the two or more of the hoses of the dry fogging system are different (e.g., are different colors, different visual indicators). In some examples, the hose to provide liquid from the tank to the spray device is translucent (e.g., clear plastic hose) to provide a visible indicator whether there is liquid in the hose. In some examples, the hose from the control device to the tank is a first color (e.g., dark blue), the hose from the control device to the spray device is a second color (e.g., light blue), and the hose from the tank to the spray device is a third color (e.g., clear, translucent, etc.). The different hoses having different visible characteristics (e.g., color, clear, etc.) allows the hoses to be used properly without confusion. In some embodiments, each of the different hoses has a different type of connector (e.g., different size, etc.) to prevent incorrect routing of hoses between portions of the dry fogging system.

In some embodiments, the dry fogging system of the present disclosure provides rapid, precise control of the spray (e.g., atomizing application), portability of the dry fogging system, quick switching of chemicals with infinite number of tanks that can be quickly exchanged.

In some embodiments, the dry fogging system has a tank that is remote from the control device. Instead of stopping the fogging process to refill tanks and start the machine back up, the process of switching tanks is done by quick connect fittings (e.g., without stopping the compressors, etc.).

In some embodiments, the dry fogging system is portable (e.g., configured to be transported by a user into an indoor space) with shorter hoses that a conventional vehicle-mounted system. The shorter hoses save time used to stop fogging and/or to switch chemicals. The dry fogging system of the present disclosure allows fogging moisture to be adjusted at a close distance compared to conventional vehicle-mounted systems where a user is to travel between the room and the vehicle to make any adjustments. In some embodiments, the dry fogging system of the present disclosure allows multiple spray devices to be branched out so that multiple areas can be treated from the same control unit with limited personnel.

In some embodiments, the dry fogging system has a control device located closer to the spray devices than a conventional vehicle-mounted system (e.g., shorter hoses). By having the control device located closer to the spray devices, the pressure remains more accurate and produces better results (e.g., more even distribution of the chemical, cleaner areas, less time to clean an area, etc.). In some embodiments, the dry fogging system can be turned on (e.g., spray output from the spray device) by actuating the control device and/or the spray device (e.g., via corresponding valves).

The systems, devices, and methods disclosed herein have advantages over conventional solutions. The dry fogging system uses less energy, has less wasted product, and takes less time to clean (e.g., disinfect, sterilize, etc.) an area than conventional solutions. The dry fogging system provides a more controllable output (e.g., size of particles in spray, flow rate of spray, turning on and off the spray, etc.) than conventional solutions. The dry fogging system provides a cleaner area in less time by using less energy than conventional solutions. The dry fogging system has less maintenance and replacement of parts compared to conventional solutions. The dry fogging system can decrease the spread of pathogens in an area and decrease illness of users that use the area compared to conventional solutions.

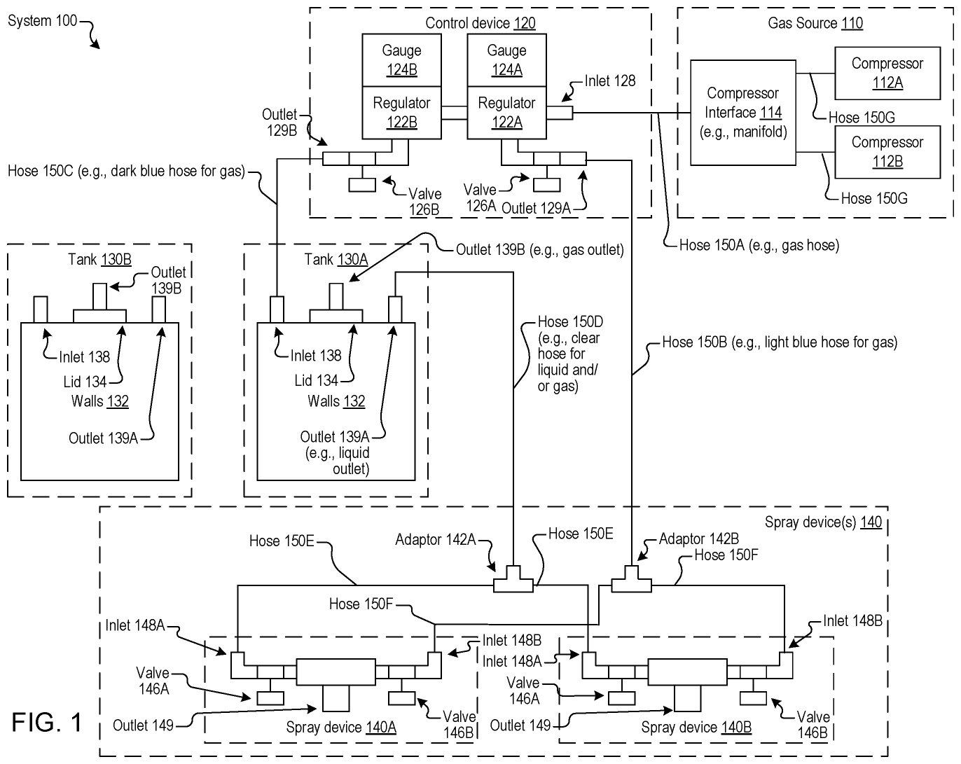

FIG. 1 illustrates a system 100, according to certain embodiments. In some embodiments, the system 100 is one or more of a dry fogging system, an atomizing system, a cleaning system, a fogging system, a sterilizing system, a disinfecting system, a sanitizing system, or the like.

System 100 includes a gas source 110, control device 120, one or more tanks 130, and one or more spray devices 140. The system 100 includes hoses 150 (e.g., pipes, tubes, etc.) that connect portions of the system 100. In some embodiments, the gas source 110, control device 120, tank 130, and spray device(s) 140 are independent (e.g., remote, separate, etc.) from each other.

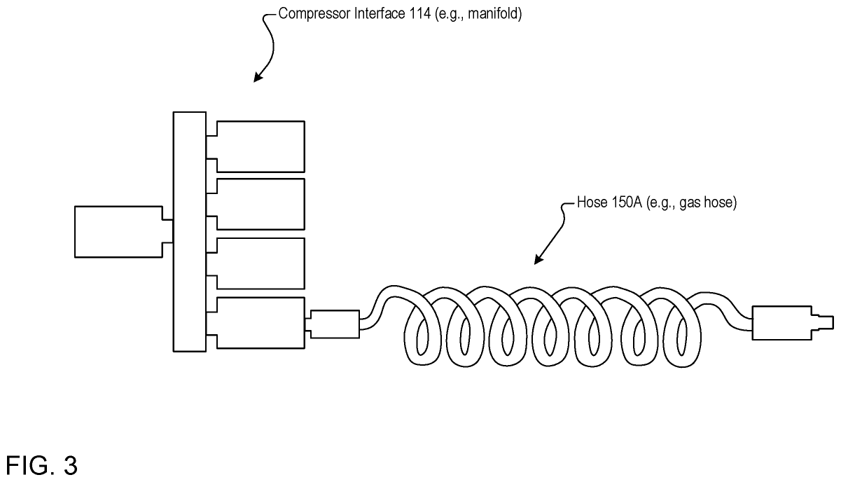

A hose 150A (e.g., see FIG. 3) connects the gas source 110 to the control device 120. A hose 150B connects control device 120 to one or more spray devices 140. A hose 150C connects the control device 120 to tank 130A. A hose 150D connects the tank 130A to the spray device(s) 140.

In some embodiments, different hoses 150 have different characteristics (e.g., visual characteristics, color, size, fitting, connectors, etc.) to prevent incorrectly connecting the system 100 via the hoses 150. Hose 150A (e.g., gas hose) provides pressurized gas from the gas source 110 to the control device 120. Hose 150B provides regulated gas (e.g., lower pressure than the pressurized gas provided by hose 150A) to the spray device(s) 140. Hose 150C provides regulated gas (e.g., lower pressure than the regulated gas provided by hose 150B) to the tank 130A. Hose 150D provides pressurized liquid from tank 130A to the spray device(s) 140. In some embodiments, the gas source 110 includes one or more compressors 112 (e.g., compressors 112A-B) and a compressor interface 114 (e.g., manifold). A corresponding hose 150G connects each compressor 112 with the compressor interface 114. In some embodiments, each of the compressors 112 provides pressurized gas at substantially the same pressure (e.g., each compressor 112 has substantially the same capacity). In some embodiments, compressor 112A provides pressurized gas at a different pressure than compressor 112B (e.g., compressors 112A and 112B have different capacities). In some embodiments, each of the hoses 150G connecting a corresponding compressor 112 with the compressor interface 144 has substantially the same properties (e.g., fluid conductance, length, inside diameter, pressure loss, etc.). In some embodiments, two or more of the hoses 150G connecting a corresponding compressor 112 with the compressor interface 144 has different properties (e.g., fluid conductance, length, inside diameter, pressure loss, etc.) than each other. The compressor interface 114 combines the pressurized gas from each of the compressors 112 into hose 150A to provide the combined pressurized gas to the control device 120.

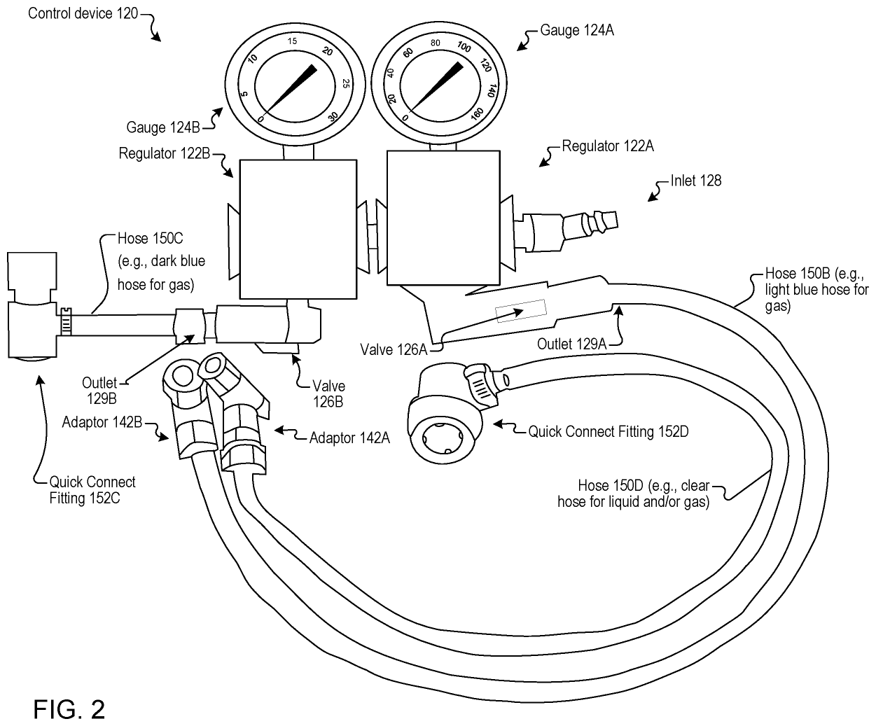

The control device 120 (e.g., see FIG. 2) includes an inlet 128 (e.g., pressurized gas control device inlet) that is coupled to the gas source 110 via hose 150A. The control device 120 includes an outlet 129A (e.g., first pressurized gas control device outlet) that is coupled to the spray device(s) 140 via the hose 150B. The control device 120 includes an outlet 129B (e.g., second pressurized gas control device outlet) that is coupled to the tank 130A via the hose 150C.

In some embodiments, the control device 120 includes a regulator 122A that receives the pressurized gas from the gas source 110 via the inlet and provides regulated gas to the outlet 129A. A valve 126A (e.g., shut-off valve) is coupled between the regulator 122A and the outlet 129A. Properties of the gas (e.g., fluid conductance, flow, pressure, etc.) provided via the outlet 129A can be adjusted via the regulator 122A and/or the valve 126A.

In some embodiments, the control device includes a regulator 122B. The regulator 122A provides regulated gas to regulator 122B (e.g., via an outlet of the regulator 122A and an inlet of regulator 122B). In some embodiments, the regulated gas provided from the regulator 122A to the regulator 122B is at a substantially same pressure (e.g. lower than the pressure of the pressurized gas received by the regulator 122A from the gas source 110) as the regulated gas provided from the regulator 122A to the outlet 129A. The regulator 122B provides regulated gas to outlet 129B (e.g., at a lower pressure than the regulated gas received by the regulator 122B. A valve 126B (e.g., shut-off valve) is coupled between the regulator 122B and the outlet 129B. Properties of the gas (e.g., fluid conductance, flow, pressure, etc.) provided via the outlet 129B can be adjusted via the regulator 122B and/or the valve 126B.

In some embodiments, the control device 120 include one or more user interfaces (e.g., gauge 124, graphical user interface, light emitting diode, touch pad, buttons, dials, etc.) to view and/or control the pressure of the regulated gas provided by the regulators 122A-B. In some embodiments, a gauge 124A is coupled to the regulator 122A to display (e.g., via a visual representation, a user interface, a needle, a light emitting diode (LED) display), etc.) the pressure of the regulated gas provided by the regulator 122A. In some embodiments, a gauge 124B is coupled to the regulator 122B to display (e.g., via a visual representation, a user interface, a needle, a light emitting diode (LED) display), etc.) the pressure of the regulated gas provided by the regulator 122B.

In some embodiments, the control device 120 has one or more regulators 122 that each provide regulated gas to one or more tanks 130 and/or one or more spray devices 140.

In some embodiments, the control device 120 includes a single regulator 122 that provides regulated gas to one or more tanks 130 and one or more spray devices 140.

In some embodiments, the control device 120 includes two regulators 122A-B, wherein the first regulator 122A provides first regulated gas to the one or more spray devices 140 and the second regulator 122B provides second regulated gas (e.g., at a lower pressure than the first regulated gas) to the one or more tanks 130.

In some embodiments, the control device 120 includes three regulators 122, where a first regulator 122 (e.g., regulator 122A) provides first regulated gas to the one or more spray devices 140, a second regulator 122 (e.g., regulator 122B) provides second regulated gas to one or more tanks 130 (e.g., tank 130A), third regulator 122 (not shown) provides third regulated gas to one or more additional tanks (e.g., tank 130B). The three regulators 122 allow powering both tank 130A and tank 130B at different pressures to apply two different chemical agents at the same time. In some embodiments, the third regulator receives regulated gas from the regulator 122B (e.g., the third regulator provides regulated gas to tank 130B at a lower pressure than the regulated gas provide by regulator 122B to tank 130A). In some embodiments, the regulator 122A tees to the regulator 122B and the third regulator so that both the regulator 122B and the third regulator receive regulated gas at substantially the same pressure from regulator 122A. This allows both regulator 122B and the third regulator to have independent control of the pressure as long as it is less than the pressure of the regulated gas provided by the regulator 122A.

In some embodiments, the control device 120 includes any number of regulators 122 and the system 100 includes any number of tanks 130 and spray devices 140, where the liquid (e.g., different chemical agents) from two or more of the tanks 130 can be provided simultaneously (e.g., via corresponding regulator 122 and/or spray device(s) 140) and/or consecutively (e.g., via the same regulator 122 and/or spray device(s) 140).

In some embodiments, the gas source 110 is located in a first location (e.g., outside of the room, outside of the building) remote from a second location (e.g., inside the room, in a support structure that supports one or more tanks 130, etc.) where the control device 120 is located. In some embodiments, the control device 120 is located proximate the one or more tanks 130 and/or the spray device(s) 140.

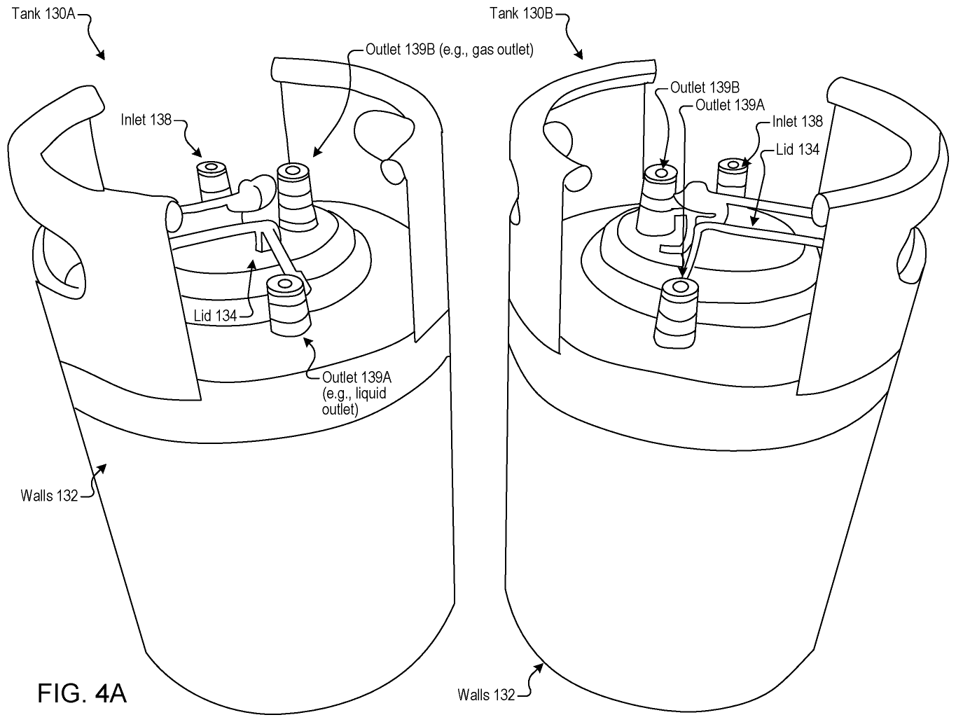

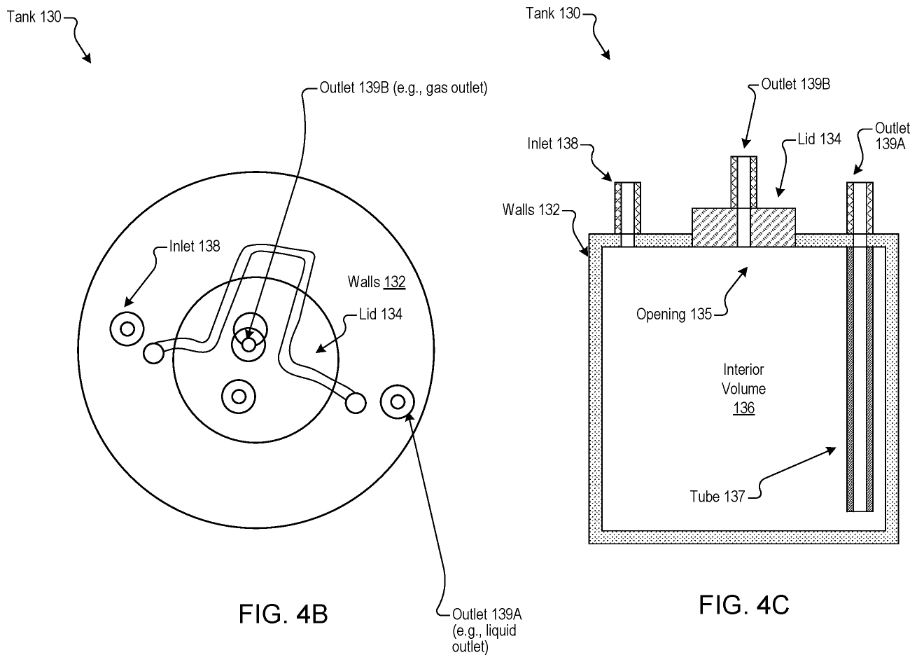

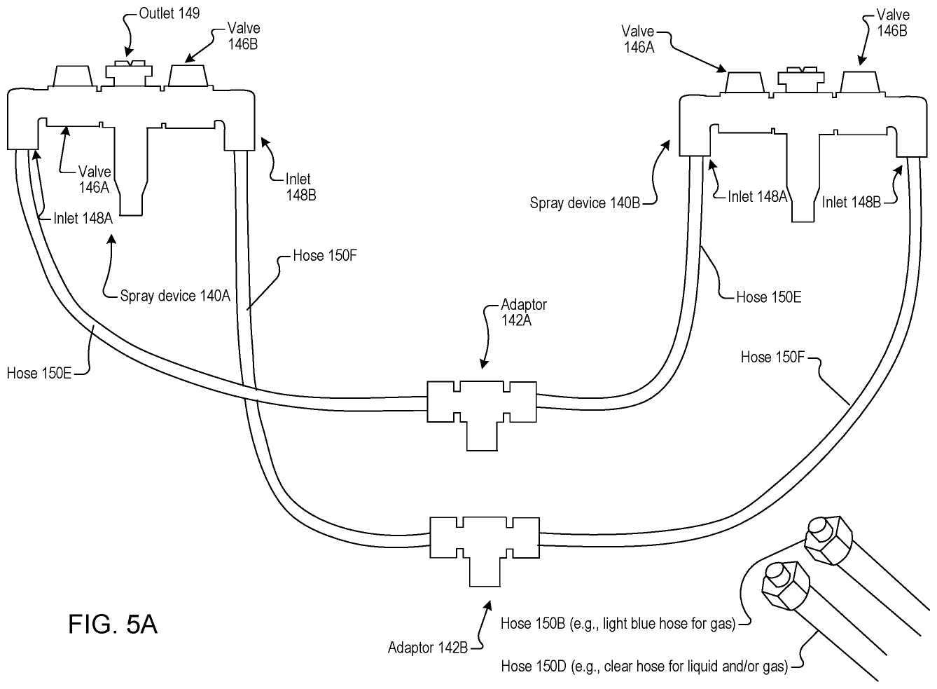

Each tank 130 (e.g., see FIGS. 4A-C) includes walls 132 that partially enclose an inner volume. The walls 132 form an opening to allow liquid to be provided into the inner volume. A lid 134 of the tank 130 is configured to engage with the walls 132 at the opening to seal the inner volume (e.g., provide a sealed environment in the tank 130). The tank 130 includes an inlet 138 (e.g., pressurized gas tank inlet), outlet 139A (e.g., pressurized liquid tank outlet), and/or outlet 139B (e.g., pressurized gas tank outlet). The inlet 138 (e.g., attached to a wall 132 of the tank 130) receives regulated gas from the control device 120 (e.g., from the regulator 122B) via hose 150C. The outlet 139A (e.g., attached to a wall 132 of the tank 130) is coupled to a tube disposed within the inner volume of the tank 130 (e.g., see FIG. 4C). The tube extends from the outlet 139A to a location proximate the bottom of the tank 130. The outlet 139A provides pressurized liquid to the spray devices 140 via the hose 150D. The hose 150D is removably attachable to the outlet 139A and to the outlet 139B. The outlet 139B (e.g., attached to the lid 134) provides pressurized gas from the tank 130 to the spray device(s) 140 to clear the liquid out of the hose 150D and the spray device(s) 140.

In some embodiments, the spray device(s) 140 includes two or more spray devices 140 and adaptors 142A-B (e.g., fittings, tees, branch point, etc.). The adaptor 142A receives pressurized liquid (e.g., responsive to the hose 150D being coupled to outlet 139A) or pressurized gas (e.g., responsive to the hose 150D being coupled to outlet 139B) from the tank 130 via hose 150D and provides the pressurized liquid or pressurized gas to the inlet 148A of one or more spay devices 140 via corresponding hoses 150E. In some embodiments, the hoses 150E are substantially similar to the hose 150D. The adaptor 142B receives regulated gas from the control device 120 (e.g., regulator 122A) via hose 150B and provides the regulated gas to the inlet 148B of one or more spay devices 140 via corresponding hoses 150F. In some embodiments, the hoses 150F are substantially similar to the hose 150B.

Each spray device 140 has an inlet 148A (e.g., first spray device inlet) that connects to hose 150E (or hose 150D) to receive pressurized liquid (e.g., responsive to the hose 150D being coupled to outlet 139A) or pressurized gas (e.g., responsive to the hose 150D being coupled to outlet 139B) from the tank 130. Each spray device 140 has an inlet 148B (e.g., second spray device inlet) that connects to hose 150F (or 150B) to receive regulated gas from the control device 120 (e.g., regulator 122A). Each spray device 140 has an outlet 149 (e.g., atomizing head, nozzle, dry fog head, spray device outlet, etc.) to provide spray (e.g., atomized chemical agent). In some embodiments, each spray device 140 has a valve 146A to control properties (e.g., fluid conductance, flow, pressure, etc.) of the pressurized liquid provided to the outlet 149 and a valve 146B to control properties (e.g., gas conductance, flow, pressure, etc.) of the regulated gas provided to the outlet 149. The pressurized liquid from the tank 130 and the regulated gas from the control device 120 (e.g., regulator 122A) mix in the spray device 140 and exit via the outlet. In some embodiments, the spray device 140 uses the Venturi effect (e.g., reduction of fluid pressure) caused by the flow of the regulated gas through the spray device 140 to cause the fluid to be extracted (e.g., from hose 150E, hose 150D, tank 130, etc.) and/or output via outlet 149. For example, as the regulated gas flows through the spray device 140, the resulting reduction in fluid pressure in the spray device pulls the liquid through the spray device 140 and through the outlet 149.

In some embodiments, one or more of the gas source 110 (e.g., compressor(s) 112), regulator 122A, regulator 122B, valve 126A, valve 126B, valve 146A, valve 146B, or outlet 149 are adjusted to provide different spray properties. In some embodiments, increased pressure of the regulated gas provided to the inlet 148B of the spray device 140 from the control device 120 (e.g., regulator 122A) provides dryer spray with smaller particle sizes (e.g., diameter of about 5-10 nanometers (nm)). In some embodiments, increased pressure of the regulated gas provided to the tank 130 from the control device 120 (e.g., regulator 122B) provides spray with larger particles (e.g., diameter greater than 10 nm). In some embodiments, increased pressure of the regulated gas provided to the inlet 148B of the spray device 140 from the control device 120 (e.g., regulator 122A) is advantageous for a first type of chemical agent and increased pressure of the regulated gas provided to the tank 130 from the control device 120 (e.g., regulator 122B) is advantageous for a second type of chemical agent.

In some embodiments, the system 100 uses at least one chemical agent that is a green chemical agent (e.g., leaves no toxic residues, EPA approved green chemicals, EPA approved cold sterilants) that is safe for users and occupants of the area. In some embodiments, the system 100 provides the chemical agent as a dry application where small particles (e.g., less than 10 nm in diameter) penetrate everywhere microorganisms and agents (e.g., pathogens, viruses, bacteria, bacterial spores, mold, mold spores, etc.) can. The particles bounce while searching out microorganisms and agents. The chemical agent breaks down cells of pathogens making the pathogens inert (e.g., no possibility of creating resistance). In some embodiments, the system 100 can be used for different types of spaces, such residential buildings (e.g., apartments, villas, houses, etc.), commercial buildings (e.g., warehouses, daycares, gyms, office spaces, etc.), healthcare buildings (e.g., hospitals, doctors offices, dentist offices, etc.), and/or the like. In some embodiments, the system 100 sterilizes an entire area (e.g., room, space, airspace, surfaces, etc.) and the corresponding heating ventilation and air conditioning (HVAC) system (e.g., ducting, etc.).

In some embodiments, the system 100 is used to apply any number of sterilants, cleaning agents, and/or protective chemicals (e.g., via multiple tanks 130). In some embodiments, system 100 is used to apply (e.g., via an atomized spray) a first chemical agent (e.g., Environmental Protection Agency (EPA) N-List of sterilants that kills all microbes including COVID-19 from surfaces, in spaces, and airspace; effective until an area is recontaminated) from tank 130A and then the system 100 is used to apply (e.g., via an atomized spray) a second chemical agent (e.g., lasts on surfaces for 90 days or more to kill new microbes that fall on the surfaces; coats surfaces to keep the surfaces sterile) from tank 130B. In some embodiments, the system 100 fills an area with the first chemical agent for about 10-20 minutes and then the system 100 fills the area with the second chemical agent for less time than it filled the area with the first chemical agent. The breakdown of the first chemical agent may be water and acetic acid. The second chemical agent may be colorless and odorless once applied. In some embodiments, the first chemical agent is approved by the Food and Drug Administration (FDA) and EPA for use on meats, fruits, and vegetables as a sterilant and food spoilage inhibitor, can be used in water purification, and can be used in hospitals to sterilize equipment between patients. In some embodiments, the second chemical agent is approved safe for use on food preparation surfaces. In some embodiments, the second chemical agent is a nano-molecule that coats surfaces. The first and second chemical agents are safe for plants, pets, and children.

In some embodiments, the system 100 uses one or more products (e.g., one or more chemical agents) and/or one or more processes (e.g., applying the one or more chemical agents) relating to COVID-19 (e.g., killing COVID-19 from the airspace and surfaces of an area) that is subject to an applicable FDA approval for COVID-19 use.

In some embodiments, the system 100 includes a single spray device 140 (e.g., no adaptor 142, no hose 150E, and no hose 150F). In some embodiments, system 100 includes two spray devices 140A-B, adaptors 142A-B, two hoses 150E, and two hoses 150F. In some embodiments, system 100 includes more than two spray devices 140, adaptors 142A-B (e.g., that have at least as many outlets as there are spray devices 140), as many hoses 150E as there are spray devices 140, and as many hoses 150F as there are spray devices 140.

In some embodiments, system 100 has multiple tanks 130 and hoses 150C-D are removably attachable to each of the tanks 130. In some examples, tank 130A houses a first chemical agent (e.g., chemical product, rinsing agent, etc.) and tank 130B houses a second chemical product (e.g., chemical product, rinsing agent, etc.). In some embodiments, tanks 130A-B house the same chemical product (e.g., chemical product, rinsing agent, etc.). In some embodiments, tank 130 and the hoses 150C-D connect via a quick connect fitting. In some embodiments, hoses 150C-D are disconnected from tank 130A and connected to tank 130B without interrupting operations of the system 100. In some examples, hoses 150C-D are disconnected from tank 130A and connected to tank 130B without stopping the gas source 110 (e.g., without stopping the compressors), without clearing out hose 150D, without turning off the valves 126A-B and/or valves 146A-B, and/or the like.

In some embodiments, spray devices 140A-B are able to clean (e.g., sanitize) about 400 cubic feet in an hour (e.g., 50 square feet room with an eight foot ceiling in an hour). In some embodiments, the spray devices 140A-B use about 1.4 gallons per hour. In some embodiments, the system 100 (e.g., with two spray devices 140) has about 5 standard cubic feet per minute (SCFM) airflow at 90 pounds pressure per spray device 140 (e.g., fogger head). In some embodiments, system 100 can be used with different airflows (e.g., from gas source 110) and/or system 100 can be used to clean different volumes of areas based on the type of spray device(s) 140 (e.g., atomizing devices, fogger heads). In some embodiments, the spray device 140 dictates the pressures and air flows (e.g., based on manufacturer recommendations). In some embodiments, the system 100 has two fog heads (e.g., two spray devices 140 with one outlet 149 per spray device, one spray devices 140 that has two outlets 149). In some embodiments, the system 100 has four fog heads (e.g., four spray devices 140 with one outlet 149 per spray device, two spray devices 140 with two outlets 149 per spray device 140). In some embodiments, the system 100 has two quick-trade tanks (e.g., tanks 130A-B that connect to hoses 150C-D via quick connect fitting). The quick connect fitting (e.g., of the hoses 150C-D and/or tanks 130) allow for easy tank 130 switching without stopping system 100 for mixing, refilling, and/or changing chemical configuration.

In some embodiments, each tank 130 has about 2.5 gallon capacity (e.g., each tank 130 houses about 2.5 gallons of chemical product). In some embodiments, each tank 130 has about 5 gallon capacity (e.g., each tank 130 houses about 5 gallons of chemical product). In some embodiments, each tank 130 has about 2.5 to about 5 gallon capacity (e.g., each tank 130 houses about 2.5 to about 5 gallons of chemical product).

In some embodiments, one or more of hoses 150A-F are dual bonded chemical and/or air pressure hose (e.g., about 50 feet length). In some embodiments, hose 150D and hose 150B are dual bonded hoses (e.g., hoses that are connected to each other). In some embodiments, hoses 150G are dual bonded hoses. In some embodiments, hoses 150E-F are dual bonded hoses.

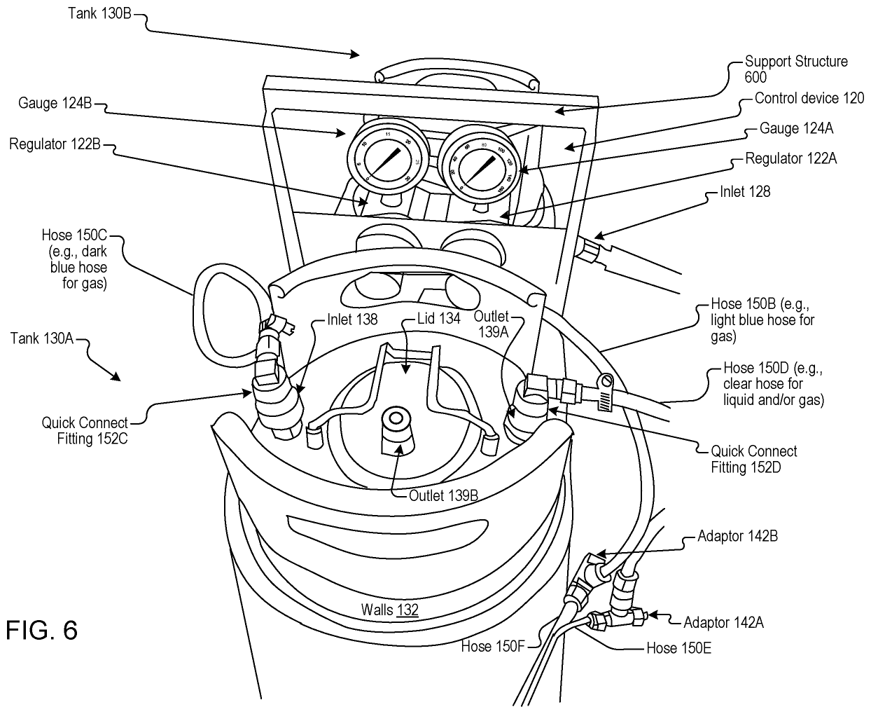

In some embodiments, the control device 120 and tank 130 are configured to be placed in a support structure (e.g., backpack) to allow walk-through fogging (e.g., allows portability, see FIG. 6).

The hose 150A (e.g., compressed air hose) from the compressor interface 114 to the control device 120 (e.g., control panel, regulators) allows the pressures to be managed (e.g., by the user) while close to the spray devices 140 (e.g., to allow the user to monitor the work).

The spray devices 140 (e.g., fog heads) are portable (e.g., configured to be transported by a user into an indoor space) to allow work in a single area (e.g., twice as fast) or to spray multiple areas at once. Additional heads (e.g., additional spray devices 140, additional outlets 149 per spray device 140) can be added to bring fog to a particular concentration faster.

In some embodiments, the system 100 starts with compressed air from one or multiple sources (e.g., gas source 110, compressors 112A-B). The compressed air combines in a compressor interface 114 (e.g., manifold) that accepts multiple inputs with one output. The compressor interface 114 is attached by a variable length compressed air hose (e.g., hose 150A, coiled hose) that leads to the control device 120 (e.g., portable control unit). The control device 120 accepts the compressed air and passes it through a regulator 122A (stage 1). Regulator 122A is coupled to a gauge 124A to read the pressure and a valve 126A (e.g., shut-off valve) before continuing on to a branch in the hose 150B. The hose 150B supplies one or more spray devices 140 (e.g., atomizers) with gas. Regulator 122B (stage 2) is connected to regulator 122A. Regulator 122B has a gauge 124B and an outlet 129B with a valve 126B (e.g., shut-off valve). Regulator 122B provides a pressure that is less than the pressure provided by regulator 122A. Regulator 122B is used to control the liquid pressure. Hose 150C connects to the outlet 129B to the tank 130 via a quick connect fitting. The control device 120 can be mounted in a carrier or removed for added mobility.

The tank 130 contains liquid that is to be atomized. The tank 130 includes an inlet 138 and two outlets 139A-B. In some embodiments, the inlet 138, outlet 139A, and outlet 139B each have a corresponding quick connect post. The outlet 139A pulls from the bottom of the tank 130. The outlet 139B pulls air from the top of the tank 130 and is used to clear the hose 150D (e.g., and adaptor 142A, hoses 150E, and/or spray device 140). The tank posts, both inlet 138 and outlets 139A-B are form specific. An inlet quick connect fitting (e.g., corresponding to inlet 138) cannot be connected to an outlet post (e.g., corresponding to outlet 139A or 139B). Only inlet quick connect fitting (e.g., of hose 150C) connects to an inlet post of inlet 138 and only outlet quick connect fitting (e.g., of hose 150D) connects to an outlet post of outlet 139A and outlet post of outlet 139B. From the outlet post of outlet 139A, liquid goes through hose 150D with the possibility to be branched to multiple spray devices 140 (e.g., atomizing heads).

Adjacent to each outlet 149 (e.g., atomizing head) is a valve 146A (e.g., shut-off) for the liquid supply and a valve 146B (e.g. shut-off) for the air supply. In some embodiments, the spray devices 140 (e.g., atomizers) are placed in a mobile stand that allows the spray devices 140 to be moved from area to area.

FIG. 2 illustrates a control device 120 of a system 100, according to certain embodiments. In some embodiments, features of FIG. 2 that have similar reference numbers as features in FIG. 1 include the same or substantially the same structure and/or functionality.

Inlet 128 of control device 120 is removably attachable to hose 150A (see FIG. 1) to receive pressurized air from a gas source 110 (see FIG. 1). Outlet 129A is connected to hose 150B and outlet 129B is connected to hose 150C.

In some embodiments, a first distal end of hose 150B is connected to outlet 129A and a second distal end of hose 150B is connected to (e.g., includes) an adaptor 142B.In some embodiments, a first distal end of hose 150C is connected to outlet 129B and a second distal end of hose 150C is connected to (e.g., includes) a quick connect fitting 152C. In some embodiments, a first distal end of hose 150D is connected to (e.g., includes) has a quick connect fitting 152D and a second distal end of hose 150D is connected to (e.g., includes) an adaptor 142A.

In some embodiments, hoses 150B, 150C, and 150D have different properties (e.g., different color, different size, different identifier, etc.). In some embodiments, quick connect fitting 152C and quick connect fitting 152D have different properties (e.g., different sizes, different identifiers). In some embodiments, adaptor 142A and adaptor 142B have different properties (e.g., different sizes, different identifiers). The different properties of different components prevents connecting components incorrectly.

FIG. 3 illustrates a compressor interface 114 of a system 100, according to certain embodiments. In some embodiments, features of FIG. 3 that have similar reference numbers as features in FIG. 1 and/or FIG. 2 include the same or substantially the same structure and/or functionality. In some embodiments, the compressor interface 114 has multiple compressor connections (e.g., multiple inlets to each couple to a corresponding compressor 112). The outlet of the compressor interface 114 is to couple to the control device 120. In some embodiments, the hose 150A is coiled.

FIG. 4A illustrates a front perspective view of tanks 130A-B of a system 100, according to certain embodiments. FIG. 4B illustrates a top perspective view of a tank 130 of a system 100, according to certain embodiments. FIG. 4C illustrates a front cross-sectional view of a tank 130 of a system 100, according to certain embodiments. In some embodiments, features of one or more of FIGS. 4A-C that have similar reference numbers as features in FIGS. 1, 2, and/or 3 include the same or substantially the same structure and/or functionality.

In some embodiments, the inlet 138 is an inlet post that is configured to couple to the quick connect fitting 152C of hose 150C (see FIG. 2). In some embodiments, the outlet 139A is an outlet post that is configured to couple to the quick connect fitting 152D of hose 150D (see FIG. 2). In some embodiments, the outlet 139B is an outlet post that is configured to couple to the quick connect fitting 152D of hose 150D (see FIG. 2).

In some embodiments, during use of system 100 (e.g., during dry fogging, during operation of the compressors 112), the quick connect fitting 152D is removed from outlet 139A of tank 130A (e.g., which stops pushing liquid through hose 150D), the quick connect fitting 152D is attached to the outlet 139A of tank 130B, the quick connect fitting 152C is removed from the inlet 138 of tank 130A, and the quick connect fitting 152C is attached to the inlet 138 of tank 130B (e.g., which causes pressurized liquid from tank 130B to be pushed through hose 150D). In some embodiments, after removing the quick connect fitting 152D from the outlet 139A and before attaching the quick connect fitting 152D to the outlet 139A of tank 130B, the quick connect fitting 152D is attached to outlet 139B of 130A (e.g., to push gas from an upper portion of the inner volume of the tank 130A through hose 150D) and the quick connect fitting 152D is removed from the outlet 139B of tank 130A.

In some embodiments, the quick connect fittings 152 are attachable and removable from an inlet 138 or outlet 139 without the use of tools (e.g., operated by hand, non-threaded connection, non-flanged connection, etc.). In some embodiments, one or more of the quick connect fittings 152, inlet 138, outlet 139A, and/or outlet 139B have self-sealing valves. In some embodiments, quick connect fitting 152C is removably attachable to inlet 138 and is not attachable to outlets 139A and/or 139B. In some embodiments, quick connect fitting 152D is removably attachable to outlets 139A and/or 139B and is not attachable to inlet 138.

In some embodiments, the lid 134 of tank 130 has a pressure release valve that is configured to be actuated (e.g., via user input) to release pressure from tank 130. In some embodiments, tank 130 has a locking device (e.g., clamp, etc.) configured to secure the lid 134 to the tank 130.

In some embodiments, tank 130 has one or more handles (e.g., protrusion with an opening for gripping) for transporting the tank 130.

Referring to FIG. 4C, tank 130 has walls 132 that enclose an inner volume 136. The walls form an opening 135. The lid 134 is configured to interface with the walls 132 to cover the opening and create a sealed environment. Inlet 138 (e.g., inlet post) is disposed on (e.g., attached to) the walls 132 (e.g., upper wall). Outlet 139A (e.g., liquid outlet post) is disposed on (e.g., attached to) the walls 132 (e.g., upper wall). Outlet 139B (e.g., gas outlet post) is disposed on (e.g., attached to the lid 134). The inlet 138, outlet 139A, and outlet 139B are in fluid communication with the inner volume 136. In some embodiments, each of the inlet 138, outlet 139A, and outlet 139B is in a closed positions responsive to not being connected to a corresponding quick connect fitting 152 and each of inlet 138, outlet 139A, and/or outlet 139B is in an open position responsive to being connected to a corresponding quick connect fitting 152.

Outlet 139A is coupled to a tube 137 that extends from the outlet 139A to a lower portion of the inner volume 136 (e.g., proximate the lower wall of walls 132 to provide liquid from the lower portion of the tank 130 to outlet 139A.

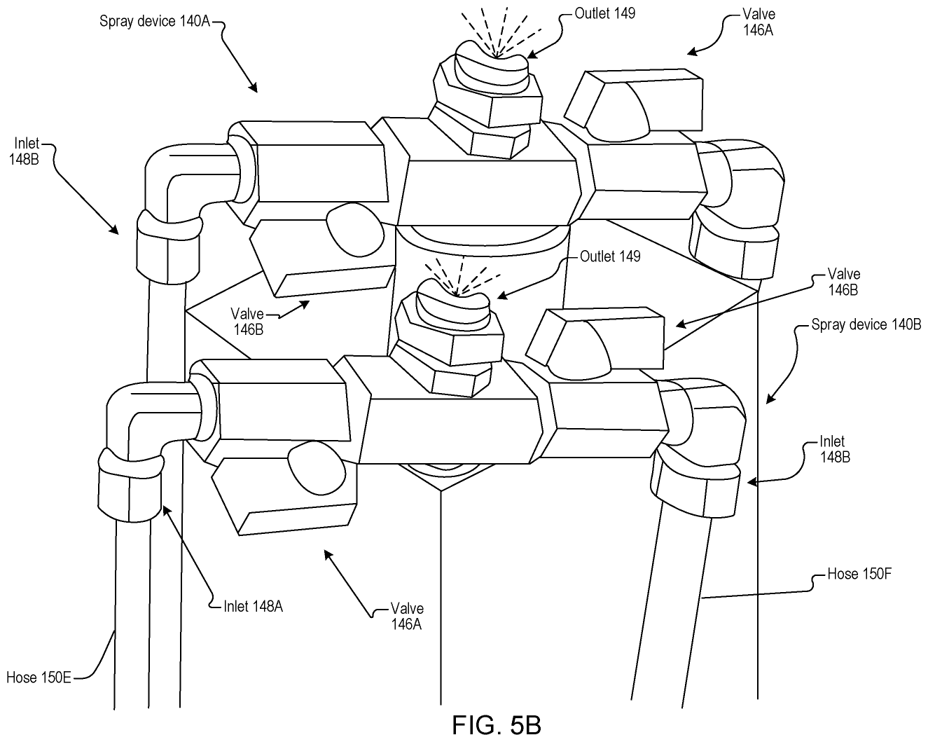

FIGS. 5A-B illustrate spray devices 140A-B of a system 100, according to certain embodiments. In some embodiments, features of one or more of FIGS. 5A-B that have similar reference numbers as features in FIGS. 1, 2, 3, 4A, 4B, and/or 4C include the same or substantially the same structure and/or functionality.

In some embodiments, the spray devices 140A-B are coupled to adaptor 142A via hoses 150E and the spray devices 140A-B are coupled to adaptor 142B via hoses 150F. In some embodiments, hoses 150E-F for spray device 140A are coupled to each other (e.g., attached with a fastener, attached with adhesive, etc.) and hoses 150E-F for spray device 140B are coupled to each other (e.g., attached with a fastener, attached with adhesive, etc.). In some embodiments, hoses 150B and 150D are coupled to each other (e.g., attached with a fastener, attached with adhesive, etc.) so that first distal ends of hoses 150B and 150D are proximate each other. In some embodiments, a first distal end of hose 150B has a first type of connector (e.g., for connecting to adaptor 142B) and a first distal end of hose 150D has a second type of connector (e.g., for connecting to adaptor 142A) that is different (e.g., different size, different shape, different type, etc.) from the first type of connector. In some embodiments, a second distal end of hose 150B has a third type of connector (e.g., for connecting to control device 120) and a second distal end of hose 150D has a fourth type of connector (e.g., quick connect fitting 152D for connecting to tank 130) that is different from the first type of connector (e.g., different size, different shape, different type, etc.).

In some embodiments, each spray device 140 has an outlet 149 (e.g., atomizing head, fog head, nozzle, etc.) on a first side of the spray device 140 and a handling portion on the second side of the spray device 140 opposite the outlet 149. In some embodiments, the handling portion of the second side of the spray device 140 is disposed in a support structure that causes the one or more spray devices 140 to be oriented to provide atomized spray into the airspace of a room (e.g., see FIG. 5B).

FIG. 6 illustrates a support structure 600 of a system 100, according to certain embodiments. In some embodiments, features of FIG. 6 that have similar reference numbers as features in FIGS. 1, 2, 3, 4A, 4B, 4C, 5A, and/or 5B include the same or substantially the same structure and/or functionality.

In some embodiments, the support structure 600 has first walls that form a first opening (e.g., cavity, enclosure, housing) to receive tank 130A and second walls that form a second opening to receive tank 130B. In some embodiments, the support structure 600 has a third wall that forms openings to secure the control device 120 (e.g., via regulators 122A-B). In some embodiments, the support structure 600 forms a handle for carrying the tanks 130A-B and control device 120.

In some embodiments, the support structure 600 has straps for carrying the support structure 600, a tank 130, and the control device 120 (e.g., on the back of a user, on the chest of the user, around the torso of a user, etc.).

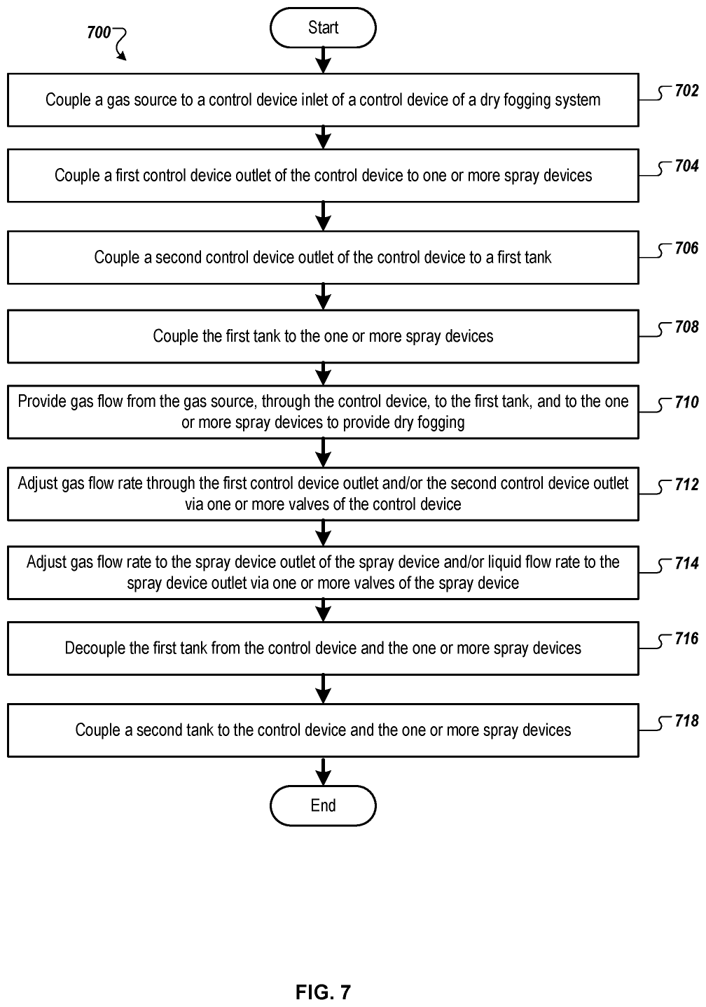

FIG. 7 illustrates a flow diagram of a method 700 of using a dry fogging system, according to certain embodiments. Although shown in a particular sequence or order, unless otherwise specified, the order of the processes can be modified. Thus, the illustrated embodiments should be understood only as examples, and the illustrated processes can be performed in a different order, and some processes can be performed in parallel. Additionally, one or more processes can be omitted in various embodiments. Thus, not all processes are required in every embodiment.

At block 702, a gas source is coupled to a control device inlet of a control device of a dry fogging system. In some embodiments, one or more compressors are coupled (e.g., connected, fluidly coupled) to a compressor interface (e.g., manifold) via corresponding hoses and the compressor interface is coupled (e.g., connected, fluidly coupled) to the control device inlet via a hose (e.g., gas hose).

At block 704, a first control device outlet of the control device is coupled to one or more spray devices of the dry fogging system. In some embodiments, at block 704, the first control device outlet is coupled (e.g., connected, fluidly coupled) to a gas adaptor via a first hose, the gas adaptor is coupled (e.g., connected, fluidly coupled) to a first spray device via a second hose, and the gas adaptor is coupled (e.g., connected, fluidly coupled) to a second spray device via a third hose. In some embodiments, the first control device outlet is coupled (e.g., connected, fluidly coupled) to a spray device via a hose (e.g., without use of an adaptor).

At block 706, a second control device outlet of the control device is coupled to a first tank. At block 706, a hose may be connected between the second control device outlet and the first tank. The hose may be connected to an inlet of the first tank via a quick connect fitting.

At block 708, the first tank is coupled to the one or more spray devices. At block 708, one or more hoses may be connected between the first tank and the one or more spray devices. In some embodiments, a first hose is coupled (e.g., connected, fluidly coupled) between the first tank and a liquid adaptor, a second hose is coupled (e.g., connected, fluidly coupled) between the liquid adaptor and a first spray device, and a third hose is coupled (e.g., connected, fluidly coupled) between the liquid adaptor and a second spray device. In some embodiments, a hose is coupled (e.g., connected, fluidly coupled) between the tank and a spray device (e.g., without use of an adaptor).

At block 710, gas flow is provided from the gas source, through the control device, to the first tank, and to the spray device to provide dry fogging. In some embodiments, block 710 is via actuation of the one or more compressors of the gas source. In some embodiments, block 710 is via actuation of the control device. In some embodiments, block 710 is via one or more of coupling of the gas source to the control device, coupling of the control device to the first tank, and/or coupling of the control device to the one or more spray devices).

At block 712, gas flow rate (e.g., and/or pressure) through the first control device outlet and/or the second control device outlet is adjusted (e.g., controlled) via one or more valves of the control device. In some embodiments, pressure via the first control device outlet and/or the second control device outlet is controlled via adjusting the first and/or second regulator.

At block 714, gas flow rate to the spray device outlet and/or liquid flow rate to the spray device outlet are adjusted (e.g., controlled) via one or more valves of the spray device. In some embodiments, a first valve of the spray device is used to control properties (e.g., fluid conductance, flow, pressure, etc.) of the pressurized liquid provided to the spray device outlet and a second valve of the spray device is used to control properties (e.g., gas conductance, flow, pressure, etc.) of the regulated gas provided to the spray device outlet.

At block 716, the first tank is decoupled from the controller device and the one or more spray devices. In some embodiments, at block 716, a hose connected between the controller device and the first tank is disconnected from the first tank (e.g., via a quick release fitting) and a hose connected between the first tank and the one or more spray devices is disconnected from the first tank (e.g., via a quick release fitting).

In some embodiments, a gas outlet of the first tank is actuated to depressurize the first tank and the first tank is opened (e.g., subsequent to being depressurized via the gas outlet) via a lid of the first tank. Liquid may be added to or removed from the first tank subsequent to being opened.

At block 718, a second tank is coupled to the controller device and the one or more spray devices. In some embodiments, at block 718, a hose is connected between the controller device and the second tank (e.g., via a quick release fitting) and a hose is connected between the second tank and the one or more spray devices (e.g., via a quick release fitting). In some embodiments, the first tank houses a first product and the second tank houses a second product and blocks 716-718 are performed to provide dry fogging with the second product. In some embodiments, the first tank and the second tank house the same product and blocks 716-718 are performed to continue dry fogging (e.g., when the first tank is depleted).

In some embodiments, blocks 716-718 are performed without turning off the gas source (are quickly performed via the quick release fittings). In some embodiments, the control device, one or more spray devices, and the one or more tanks are portable and are transported (e.g., carried) by a user into an indoor space (e.g., via a carrier, carrier structure, backpack, etc.) so that adjustments can be made to the gas and/or liquid pressure, tanks can be switched, etc. without leaving the indoor space.

The preceding description sets forth numerous specific details such as examples of specific systems, components, methods, and so forth in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular implementations may vary from these exemplary details and still be contemplated to be within the scope of the present disclosure.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

The words “example” or “exemplary” are used herein to mean serving as an example, instance or illustration. Any aspect or design described herein as “example” or “exemplary” is not necessarily to be construed as preferred or advantageous over other aspects or designs. Rather, use of the words “example” or “exemplary” is intended to present concepts in a concrete fashion.

Reference throughout this specification to “one embodiment,” “an embodiment,” or “some embodiments” means that a particular feature, structure, or characteristic described in connection with the embodiment is included in at least one embodiment. Thus, the appearances of the phrase “in one embodiment,” “in an embodiment,” or “in some embodiments” in various places throughout this specification are not necessarily all referring to the same embodiment. In addition, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Also, the terms “first,” “second,” “third,” “fourth,” etc. as used herein are meant as labels to distinguish among different elements and can not necessarily have an ordinal meaning according to their numerical designation. When the term “about,” “substantially,” or “approximately” is used herein, this is intended to mean that the nominal value presented is precise within ±10%.

Although the operations of the methods herein are shown and described in a particular order, the order of operations of each method may be altered so that certain operations may be performed in an inverse order so that certain operations may be performed, at least in part, concurrently with other operations. In another embodiment, instructions or sub-operations of distinct operations may be in an intermittent and/or alternating manner.

It is understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. A dry fogging system comprising:
a control device comprising a control device inlet configured to couple to a gas source, a first control device outlet, and a second control device outlet configured to couple to a first tank; and
a first spray device comprising a first spray device inlet configured to couple to the first tank, a second spray device inlet configured to couple to the first control device outlet, and a spray device outlet configured to provide dry fogging.

2. The dry fogging system of claim 1, wherein the control device further comprises:
a first regulator configured to receive pressurized gas at a first pressure from the gas source via the control device inlet, wherein the first regulator is further configured to provide the pressurized gas at a second pressure via the first control device outlet; and
a second regulator configured to receive the pressurized gas from the first regulator, wherein the second regulator is further configured to provide the pressurized gas at a third pressure via the second control device outlet.

3. The dry fogging system of claim 2, wherein the control device further comprises:
a first valve disposed between the first regulator and the first control device outlet; and
a second valve disposed between the second regulator and the second control device outlet.

4. The dry fogging system of claim 2, wherein the control device further comprises:
a first gauge coupled to the first regulator to display the second pressure; and
a second gauge coupled to the second regulator to display the third pressure.

5. The dry fogging system of claim 2 further comprising the first tank comprising:
a first tank inlet configured to receive the pressurized gas at the third pressure;
a first tank outlet configured to provide pressurized product; and
a second tank outlet configured to be actuated to depressurize the first tank.

6. The dry fogging system of claim 1, wherein the first spray device further comprises:
a first valve disposed between the first spray device inlet and the spray device outlet; and
a second valve disposed between the second spray device inlet and the spray device outlet.

7. The dry fogging system of claim 1 further comprising:
a gas adaptor comprising a first gas adaptor inlet configured to couple to the first control device outlet, a first gas adaptor outlet configured to couple to the first spray device, and a second gas adaptor outlet configured to couple to a second spray device; and
a liquid adaptor comprising a first liquid adaptor inlet configured to couple to the first tank, a first liquid adaptor outlet configured to couple to the first spray device, and a second liquid adaptor outlet configured to couple to a second spray device.

8. The dry fogging system of claim 1 further comprising:
a gas hose disposed between the second control device outlet and the first tank; and
a liquid hose disposed between the first tank and the first spray device, wherein the gas hose and the liquid hose are configured to be decoupled from the first tank and coupled to a second tank.

9. The dry fogging system of claim 1, wherein the control device, first tank, and first spray device are configured to be transported by a user into an indoor space via one or more of a support structure, a carrier, or a backpack.

10. A control device of a dry fogging system, the control device comprising:
a control device inlet configured to couple to a gas source to receive pressurized gas at a first pressure;
a first regulator coupled to the control device inlet to receive the pressurized gas at the first pressure and to provide the pressurized gas at a second pressure via a first control device outlet to a first spray device; and
a second regulator coupled to the first regulator to receive the pressurized gas at the second pressure and to provide the pressurized gas at a third pressure via a second control device outlet to a first tank, wherein the first tank is to provide a pressurized liquid to the first spray device.

11. The control device of claim 10 further comprising:
a first valve coupled between the first regulator and the first control device outlet, wherein the first valve is configured to provide first flow rate control of the pressurized gas at the second pressure; and
a second valve coupled between the second regulator and the second control device outlet, wherein the second valve is configured to provide second flow rate control of the pressurized gas at the third pressure.

12. The control device of claim 10 further comprising:
a first gauge coupled to the first regulator to provide a first visual representation of the pressurized gas at the second pressure; and
a second gauge coupled to the second regulator to provide a second visual representation of the pressurized gas at the third pressure.

13. The control device of claim 10, wherein the control device inlet is configured to couple to a compressor interface of the gas source, wherein the compressor interface is configured to couple to one or more compressors.

14. The control device of claim 10, wherein the first control device outlet is configured to couple to the first spray device and a second spray device via a first adaptor, and wherein the first tank is configured to couple to the first spray device and the second spray device via a second adaptor.

15. A method comprising:
coupling a gas source to a control device inlet of a control device of a dry fogging system;
coupling a first control device outlet of the control device to a first spray device of the dry fogging system;
coupling a second control device outlet of the control device to a first tank;
coupling the first tank to the first spray device; and
providing gas flow from the gas source, through the control device, to the first tank, and to the spray device to provide dry fogging.

16. The method of claim 15 further comprising one or more of:
adjusting first gas flow rate through the first control device outlet via a first valve of the control device; or
adjusting second gas flow rate through the second control device outlet via a second valve of the control device.

17. The method of claim 15 further comprising one or more of:
adjusting gas flow rate to the spray device outlet via a first valve of the spray device; or
adjusting liquid flow rate to the spray device outlet via a second valve of the spray device.

18. The method of claim 15, wherein the coupling of the second control device outlet to the first tank comprises coupling a first hose between the second control device outlet and the first tank, wherein the coupling of the first tank to the first spray device comprises coupling a second hose between the first tank and the first spray device, and wherein the method further comprises:
decoupling the first hose and the second hose from the first tank; and
coupling the first hose and the second hose to a second tank.

19. The method of claim 15 further comprising:
actuating a gas outlet of the first tank to depressurize the first tank; and
opening the first tank via a lid of the first tank.

20. The method of claim 15 further comprising:
coupling the first control device outlet to a gas adaptor;
coupling a second hose between the first tank to a liquid adaptor;
coupling the gas adaptor to the first spray device and a second spray device; and
coupling the liquid adaptor to the first spray device and the second spray device.
